(19) United States
(12) Patent Application Publication
(10) Pub. No.: US 2005/0053560 A1
(43) Pub. Date: Mar. 10, 2005

(54) Use of a composition containing extracts of Vitis vinifera and lycopersicum for protecting the skin

(75) Inventors: Orlando Petrini, Comano (CH); Inka Pfitzner, Stuttgart (DE); Juergen Bernhardt, Weinstadt (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Pharmaton S.A, Bioggio (CH)

(21) Appl. No.: 10/656,618

(22) Filed: Sep. 5, 2003

(51) Int. Cl. A61K 7/42; A61K 31/355; A61K 35/78; A61K 33/04

(52) U.S. Cl. 424/59; 424/766; 424/777; 424/702; 514/474; 514/763; 514/458

(57) ABSTRACT

The invention relates to the use of a composition containing synergistic amounts of antioxidants selected from the group consisting of: Vitamin E Beta-carotene Vitamin C Selenium
an extract of Lycopersicum esculentum
an extract of Vitis vinifera, and optionally a suitable carrier material for preparing a pharmaceutical composition, food supplement or cosmetic for protecting the skin from UV rays, ageing under the effect of UV rays and/or inflammatory processes.

### BACKGROUND TO THE INVENTION

1. Technical Field

The invention relates to the use of a composition containing synergistic amounts of various antioxidants and optionally a suitable carrier material for preparing a pharmaceutical composition, food supplement or cosmetic agent for protecting the skin from UV rays and/or inflammatory processes.

2. Prior Art

The skin is exposed to a variety of external stress factors, being the boundary layer and surface of the human body. The human skin is an organ which protects the body from external influences by means of differently specialised cell types such as the keratinocytes, melanocytes, Langerhans cells, Merkel cells and sense cells incorporated therein. A distinction must be drawn between external physical, chemical and biological influences on the human skin. The external physical influences include thermal and mechanical influences and the effects of radiation such as UV and IR radiation. The external chemical influences include in particular the effects of toxins and allergens. The external biological influences include the effects of foreign organisms and their metabolic products. Other stress factors are pathological conditions and diseases such as fever, inflammation, infection and cell and tissue trauma as well as physiological processes such as cell division. The aim of the present invention is therefore to overcome or at least alleviate the problems mentioned above and provide a method of improving the defence status of the skin cells.

European Patent Application EP 0712630 relates to an oral composition for preventing sun allergies. The composition is based on a carotenoid, a tocopherol, ascorbic acid and selenium. The carotenoids used are carotene and lycopene. EP 0712630 mentions the already known combination of antioxidants with plant oil as an adjuvant in the production of capsules for oral use. Medicinal uses and/or effects apart from the effect against sun allergy of oral administration of the composition of EP 0712630 A2 have not been disclosed.

International Patent Application WO 01/89542 describes the use of a synergistic combination of vitamin E, provitamin A, vitamin C, and extract of Lycopersicum esculentum, and an extract of Vitis vinifera, to protect the cells against radicals.

However, there is no indication that such a combination could improve the defence status of the skin cells.

### BRIEF SUMMARY OF THE INVENTION

The invention thus relates to a composition containing synergistic amounts of antioxidants selected from the group consisting of:
Vitamin E,
Provitamin A,
Vitamin C,
Selenium,
an extract of Lycopersicum esculentum,
an extract of Vitis vinifera, and
   optionally a suitable carrier for preparing a pharmaceutical composition, food supplement or cosmetic for protecting the skin from UV rays and/or inflammatory processes.

### BRIEF DESCRIPTION OF THE DRAWINGS

FIG. 1 shows the diagrammatic structure of the membrane equivalent with fibroplasts (1.1), keratinocytes (1.2) and extracellular matrix proteins (1.3).

FIG. 2 shows the diagrammatic structure of the collagen equivalent with collagen gel (2.1) and keratinocytes (1.2).

FIG. 3 shows histological sections of membrane equivalents of

A: An unsupplemented control equivalent with a differentiated keratinocyte layer (“Stratum corneum”) (3.1), a proliferating keratinocyte layer (3.2) and the membrane (3.3);

B: A supplemented control equivalent with a thickened Stratum corneum (3.4) and cutting artefacts (3.5).

FIG. 4 shows histological sections of membrane equivalents of membrane equivalents [sic] after UVA irradiation (20 J/cm²)

A: Unsupplemented membrane equivalent with a condensed cell nucleus (4.1) and a differentiated keratinocyte layer (4.2),

B: Supplemented membrane equivalent with a thickened Stratum corneum (4.3), a condensed cell nucleus (4.4) and a proliferating keratinocyte layer (4.5).

FIG. 5 shows the immune cells 24 hours after activation, immediately before “co-cultivation” with the membrane equivalents.

A: Unsupplemented immune cells;

B: Supplemented immune cells.

FIG. 6 shows the unsupplemented, immunostimulated membrane equivalent A with condensed cell nucleus (6.1) and a loss of the barrier integrity (6.2) and the supplemented immunostimulated membrane equivalent B with a thickened Stratum corneum (6.3), condensed cell nucleus (6.4) and a proliferating keratinocyte layer with loss of barrier integrity (6.5).

FIG. 7 shows haemalum/eosin staining

A: Normal skin with Stratum corneum (7.1), Stratum granulosum (7.2), Stratum spinosum (7.3) and Stratum basale (7.4);

B: Collagen equivalent with a differentiated keratinocyte layer (7.5), a flattened keratinocyte layer (7.6), a polygonal keratinocyte layer (7.7) and a cylindrical keratinocyte layer (7.8).

FIG. 8 shows haemalum/eosin staining of a collagen equivalent with a thickened Stratum corneum (8.1).

FIG. 9 shows a Tunel assay before UV irradiation A: Unsupplemented collagen equivalent with non-specific binding (9.1) and apoptotic cells (9.2); B: Supplemented collagen equivalent.

FIG. 10 shows a Tunel assay after UV irradiation

A: Unsupplemented collagen equivalent with apoptotic cells (10.1);

B: Supplemented collagen equivalent.

### DETAILED DESCRIPTION OF THE INVENTION

The composition containing synergistic amounts of antioxidants selected from the group comprising vitamin E, provitamin A, vitamin C, selenium, and extract of Lycopersicum esculentum, an extract of Vitis vinifera and optionally a suitable carrier material can be administered orally or topically to protect the skin from UV rays and/or inflammatory processes. The composition is preferably administered orally.

Tocopherols are chroman-6-ols (3,4-dihydro-2H-1-benzopyran-6-ols) substituted in the 2 position by a 4,8,12-trimethyltridecyl group and are effective as vitamin E. A distinction is made between a-, b-, g-, d- and e-tocopherol, among others, the latter still having the original unsaturated prenyl side chain, as well as a-tocoquinone and -hydroquinone, in which the pyran ring system has been opened.

The commonest and most effective natural tocopherol is α-tocopherol. It occurs in many vegetable oils, particularly seed oils from soya, wheat, maize, rice, cotton, alfalfa and nuts.

Tocopheryl acetate, succinate, nicotinate and poly(oxyethylene)succinate are the usual forms for administration as vitamin E.

Provitamin A, β-carotene, is a precursor of vitamin A, which is oxidatively cleaved in the animal body into 2 mol of retinal and reduced to retinol (vitamin A). Provitamin A is the commonest carotenoid in the plant kingdom, predominantly in the all-trans form, e.g. in carrots and crude palm oil and as an accompaniment to chlorophyll.

Vitamin C, L-ascorbic acid, {(R)-5-[(S)-1,2-dihydroxyethyl]-3,4-dihydroxy-5H-furan-2-one, is found in all higher plants and animals, particularly in acerola, citrus fruits, rosehips, sea buckthorn, strawberries, blackcurrants, spinach, peppercorns, horseradish, parsley and liver.

Using a microbiological process originally developed by Reichstein in 1934 D-glucose is first hydrogenated to form sorbitol and then this is bacterially oxidised to form L-sorbose. This ketose is converted via its bis-O-isopropylidene derivative into that of 2-oxo-L-gulonic acid and the latter is converted into L-ascorbic acid with acids.

Selenium deficiency is also associated with rheumatism and cataract; selenite is supposed to potentiate the effect of vitamin E and also detoxify the body of mercury and cadmium. The human body contains approx. 10-15 mg of selenium and becomes ill when the daily food intake contains more than 1 mg of Se/g; by contrast, a minimum content of 0.02 mg Se/g is necessary to prevent deficiency symptoms. Se is stored in the human body in the liver, spleen, kidneys and heart.

Extract of Lycopersicum esculentum,

1 kg of tomatoes contains approx. 20 mg lycopene, rosehips and other fruits where it occurs alongside its isomers, the carotenoids and the 1,2-epoxide as well as the 5,6-epoxide. Lycopene is also present in chanterelles (Cantharellus cibarius), butter, serum and liver. Lycopene is licensed as a colouring for cosmetics and food (E 160 d).

The tomatoes are extracted using known methods. The resulting standardised extract contains about 5 wt. % of lycopene, which is partly dissolved in natural lipids of the tomatoes and partly in crystalline form and dispersed in these lipids.

Extract of Vitis vinifera, red wine extract, is important because it contains resveratrol as its essential ingredient. Resveratrol is 3,5,4′-trihydroxy-stilbene. Resveratrol is present in fairly large amounts in the skin of blue wine grapes, in red wine and grape juice, in groundnuts and mulberries. Resveratrol has also been identified as the essential ingredient of “Kojo-Kon”, a popular medicine in China and Japan. Resveratrol is used to treat arteriosclerosis and counteracts the tendency of the blood platelets to clump together and reduces susceptibility to thrombosis. Red wine extract has a protective effect against arteriosclerosis and cancer, particularly on account of its content of resveratrol.

Red wine with resveratrol protects the LDL particles in the blood from oxidation significantly more than a comparable amount of vitamin E, which is also an antioxidant.

The extract of Vitis vinifera is preferably used as in U.S. Pat. No. 6,297,218 in the form of a composition containing phospholipids and vegetable oils.

Preferably, the grapeseeds are extracted with a solvent selected from the group comprising the alcohols, such as for example ethanol, propanol or butanol, ketones, such as for example acetone or methylethylketone, and water, or with a mixture of the above solvents at a temperature from 20° C. to 100° C., particularly from 40 to 80° C.

Preferably, according to the invention, the composition consists of:
2 to 15 wt. %, particularly 4 to 10 wt. %, most preferably about 6.8 wt. % vitamin E;
0.1 to 5 wt. %, particularly 0.5 to 3 wt. %, most preferably about 1.6 wt. % Provitamin A in the form of a 30% suspension;
20 to 60 wt. %, particularly 30 to 50 wt. %, most preferably about 40.7 wt. % vitamin C;
5 to 20 wt. %, particularly 7.5 to 17.5 wt. %, most preferably about 16.9 wt. % selenium in the form of a selenium yeast;
5 to 20 wt. %, particularly 7.5 to 17.5 wt. %, most preferably about 16.9 wt. % of extract of Lycopersicum esculentum, and
5 to 20 wt. %, particularly 7.5 to 17.5 wt. %, most preferably about 16.9 wt. % of extract of Vitis vinifera; based on the total amount of antioxidants.

For oral administration the composition is preferably used in the form of a soft or hard gelatine capsule, tablet or film-coated tablet.

For oral use the following carriers are preferred: natural vegetable oils, totally or partially hydrogenated vegetable oils, lecithins, plant phosphatides and natural waxes, particularly soya oil, totally or partially hydrogenated soya oil, rapeseed oil, groundnut oil, soya lecithin, soya phosphatides, egg lecithin and beeswax.

For oral administration the composition preferably consists essentially of
15 to 45 wt % of antioxidants and
55 to 85 wt % of carrier material.

Moreover, the composition according to the invention may be used in the form of suppositories or in a transdermal form.

The compositions may also be used according to the invention in the form of a topical composition.

A topical composition is prepared by incorporating the combination of the antioxidants, optionally with excipients and/or carriers, in a suitable formulation. The excipients and carriers are selected from the group of carriers, preservatives and other conventional excipients.

The topical composition based on the combination of antioxidants is applied externally to the skin or skin adnexa.

Suitable formulations include, for example: solutions, suspensions, emulsions, pastes, ointments, gels, creams, lotions, powders, soaps, cleansing preparations containing surfactants, oils and sprays. In addition to one or more combinations of antioxidants used according to the invention, any other conventional carriers, excipients and possibly other active substances may be added to the composition.

Preferred excipients are selected from among the preservatives, stabilisers, solubilisers, vitamins, colouring agents and odour improvers.

Ointments, pastes, creams and gels may contain, in addition to one or more antioxidants used according to the invention, the usual carriers such as animal and vegetable fats, waxes, paraffins, starch, gum tragacanth, cellulose derivatives, polyethylene glycols, silicons, bentonite, silicic acid, talc and zinc oxide or mixtures of these substances.

Powders and sprays may contain, in addition to one or more antioxidants used according to the invention, the conventional carriers, e.g. lactose, talc, silicic acid, aluminium hydroxide, calcium silicate and polyamide powders or mixtures of these substances. Sprays may additionally contain the usual propellants, e.g. chlorofluorohydrocarbons, propane/butane or dimethylether.

The compositions which are to be applied topically may contain organic or inorganic UV filters in addition to the antioxidants used according to the invention. Such UV filters are recommended as a supplementary protection when the composition according to the invention is administered orally.

Suitable organic UV filters include all the UVA and UVB filters known to the skilled man. For both UV ranges there are numerous tried and tested substances known from the specialist literature, e.g.

benzylidene camphor derivatives such as

3-(4′-methylbenzylidene)-dl-camphor (e.g. Eusolex® 6300),
3-benzylidenecamphor (e.g. Mexoryl® SD),
N,N,N-trimethyl-4-(2-oxoborn-3-ylidenemethyl)-anilinium-methylsulphate (e.g. Mexoryl® SK) or
alpha-(2-oxoborn-3-ylidene)toluene-4-sulphonic acid (e.g. Mexoryl® SL),
   benzoyl or dibenzoyl methanes, such as
1-(4-tert-butylphenyl)-3-(4-methoxyphenyl)propane-1,3-dione (e.g. Eusolex® 9020) or
4-isopropyldibenzoylmethane (e.g. Eusolex® 8020),
   benzophenones, such as
2-hydroxy-4-methoxybenzophenone (e.g. Eusolex® 4360) or
2-hydroxy-4-methoxybenzophenone-5-sulphonic acid and the sodium salt thereof (e.g. Uvinul® MS-40), methoxycinnamic acid esters; such as
2-ethylhexyl p-methoxycinnamate (e.g. Eusolex® 2292),
isopentyl p-methoxycinnamate, e.g. as a mixture of the isomers (e.g. Neo Heliopan® E 1000),
   salicylate derivatives, such as
2-ethylhexylsalicylate (e.g. Eusolex® OS),
4-isopropylbenzylsalicylate (e.g. Megasol®) or
3,3,5-trimethylcyclohexylsalicylate (e.g. Eusolex® HMS),
   4-aminobenzoic acid and derivatives thereof, such as
4-aminobenzoic acid,
2-ethylhexyl 4-(dimethylamino)benzoate (e.g. Eusolex® 6007),
ethoxylated ethyl 4-aminobenzoate (e.g. Uvinul® P25),
   and other substances, such as
2-ethylhexyl 2-cyano-3,3-diphenylacrylate (e.g. Eusolex® OCR),
2-phenylbenzimidazole-5-sulphonic acid and the potassium, sodium and triethanolamine salts thereof (e.g. Eusolex® 232),
3,3′-(1,4-phenylenedimethylene)-bis-(7,7-dimethyl-2-oxobicyclo[2.2.1]hept-1-ylmethanesulphonic acid and the salts thereof (e.g. Mexoryl® SX) and
2,4,6-trianilino-(p-carbo-2′-ethylhexyl-1′-oxy)-1,3,5-triazine (e.g. Uvinul® T 150).

Suitable inorganic UV filters are those selected from among the titanium dioxides, e.g. coated titanium dioxide (e.g. Eusolex® T-2000 or Eusolex® T-Aqua), zinc oxides (e.g. Sachtote®), iron oxides or cerium oxides.

Preferred UV filters are zinc oxide, titanium dioxide, 3-(4′-methylbenzylidene)-dl-camphor, 1-(4-tert. butylphenyl)-3-(4-methoxyphenyl)propan-1,3-dione, 4-isopropyldibenzoylmethane, 2-hydroxy-4-methoxybenzophenone, octyl methoxycinnamate, 3,3,5-trimethylcyclohexylsalicylate, 2-ethylhexyl 4-(dimethylamino)-benzoate, 2-ethylhexyl 2-cyano-3,3-diphenylacrylate, 2-phenylbenzimidazole-5-sulphonic acid and the potassium, sodium and triethanolamine salts thereof.

Particularly preferred UV filters are zinc oxide and titanium dioxide.

If titanium dioxide is used according to the invention it is preferable to use, in addition to the titanium dioxide, one or more other UV filters selected from 3-(4′-methylbenzylidene)-dl-camphor, 1-(4-tert-butylphenyl)-3-(4-methoxyphenyl)propan-1,3-dione, 4-isopropyldibenzoylmethane, 2-hydroxy-4-methoxybenzophenone, octyl methoxycinnamate, 3,3,5-trimethylcyclohexylsalicylate, 2-ethylhexyl 4-(dimethylamino)benzoate, 2-ethylhexyl 2-cyano-3,3-diphenylacrylate, 2-phenylbenzimidazol-5-sulphonic acid and the potassium, sodium and triethanolamine salts thereof.

It is particularly preferred to use, in addition to titanium dioxide, the UV filters 2-hydroxy-4-methoxybenzophenone and/or 2-ethylhexyl p-methoxycinnamate.

The composition to be used according to the invention is obtainable under the trade mark Seresis® and has the following ingredients:

TABLE 1
Test substances with amounts per 500 mg
Seresis ® capsule.

| Substance | Declared amount/500 mg |
|---|---|
| standardised tomato extract | 25.0 mg |
| β-Carotene suspension 30% | 2.4 mg |
| D,L-α-Tocopheryl acetate | 10.0 mg |
| standardised grapeseed extract | 25.0 mg |
| ascorbic acid | 60.0 mg |
| selenium yeast | 25.0 mg |

As a comparison the total contents of a Seresis® capsule (Batch No. 115576) were used in the cell culture experiments.

2.2 In vitro Test Systems—Cultivation and Construction

For the present cytotoxicity tests and the Fluoroscan process normal human skin fibroblasts of the cell line NHDF were used as the test system, depending on their availability.

The NHDF cells were cultivated under standardised cell culture conditions (37° C., 5% CO₂). To carry out the tests the cells were seeded in the corresponding plate formats. After confluence was achieved, i.e. after a continuous cell lawn had formed, the cells were supplemented. The histological photographs were taken with two different in vitro skin models. These models were a membrane and a collagen equivalent based on primary pooled fibroblasts and keratinocytes of the cell line HaCaT. The HaCaT cell line represents a skin keratinocyte cell line of human origin immortalised by spontaneous transformation. The use of these cells rules out inter-individual differences such as occur with primary keratinocytes, so that the models can be constructed reproducibly.

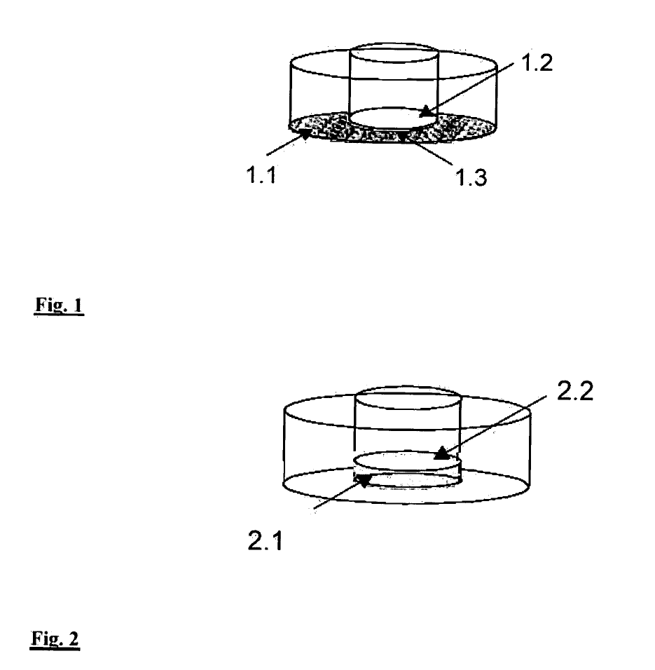

Membrane equivalent (FIG. 1): This model is a co-culture in which the cell types are cultivated in separate compartments. The exchange of substances in this case is provided by means of permeable membranes with a defined pore size. In order to construct this model, first of all primary pooled fibroblasts are seeded in a suitable plate format and cultivated to confluence. Immediately before the keratinocyte cell line HaCaT is seeded out in the inserts the membranes of the Transwell-Clear inserts used (made by Corning, Action, Mass.) are coated with extracellular matrix proteins. The HaCaTs are kept submerged until confluent and then maintained as an airlift culture for up to 20 days to ensure differentiation of the epidermis. Thanks to the properties of the membrane the construction of this model can easily be monitored by microscopy. A further advantage of this model is that the keratinocytes are in direct contact with the culture medium over the underside of the membrane and can thus react directly to the supplementation and stress induction.

Collagen Equivalent (FIG. 2): This model differs from the membrane equivalent described above by its proportion of dermis. The dermis is formed from fibroblasts embedded in a collagen matrix. After a short cultivation period the keratinocytes are seeded on to the collagen/fibroblast gel, kept submerged for about two days and then maintained as an airlift culture for 12-15 days. With this model the in vivo situation can be simulated better than with the membrane equivalent. However, with systemic application of the supplements or the stressors, these have to overcome the dermis part before reaching the keratinocytes. Consequently, in this model, the supplementation period is longer.

2.3 Stock Solutions of the Active Substances

As the six test substances have different solubility characteristics on account of their chemical structures, different stock solutions were prepared which were then added to the cell culture medium in corresponding dilutions (cf. 2.5, 2.6, 2.7). The tomato extract (lycopene), the ∃ carotene suspension and ∀-tocopheryl acetate were dissolved in THF and the other active ingredients were dissolved in aqueous cell culture medium or buffer. The quantity of organic solvent added to the cell culture was not more than 0.1%.

Table 1 shows the solubilisers used for the individual substances and the concentrations of the substances in the stock solutions:

TABLE 2
Solubilisers and concentrations of the
individual substances in the stock solution.

| | | Concentration | Concentration |
|---|---|---|---|
| Solubiliser | Substances | mg/mL | mM |
| THF | Standardised Tomato | 50.0 | 4.7 |
| | Extract | | (Lycopene) |
| | β-Carotene | 4.8 | 2.7 |
| | Suspension 30% | | |
| | D,L-α-Tocopheryl | 20.0 | 42.3 |
| | acetate | | |
| Medium/ | Standardised Grape | 50.0 | — |
| Buffer | Seed Extract | | |
| | Ascorbic Acid | 120.0 | 681.4 |
| | Selenium Yeast | 50.0 | — |

The contents of the Seresis® capsule were dissolved in THF and further diluted with medium in order to dissolve all the ingredients of the capsule as fast as possible. Table 3 lists the concentrations of the active ingredients in the capsule stock solution:

TABLE 3
Concentrations of the individual substances
in the Seresis ® capsule stock solution.

| Substance | Concentration in mg/mL |
|---|---|
| Standardised Tomato Extract | 2.5 |
| β-Carotene Suspension 30% | 0.24 |
| D,L-α-Tocopheryl acetate | 1.0 |
| Standardised Grape Seed Extract | 2.5 |
| Ascorbic Acid | 6.0 |
| Selenium Yeast | 2.5 |

2.6 Supplementation and Stress Treatment of the In Vitro Skin Models

The supplementation of the collagen equivalents was carried out 5 days before and that of the membrane equivalents 48 hours before the stress treatment. In both cases the dilution of the stock solutions used (individual substances) was 1:1000. The discrepancy in the supplementation period can be explained, as described above, by the accessibility of the keratinocytes. In the case of the collagen equivalent the active substances have to pass through the collagen gel to reach the keratinocytes, with the result that a longer supplementation phase is required here. Table 4 shows the supplementation and treatment plan for the individual in vitro skin models.

In all the treatment groups, unsupplemented equivalents were used as well as the supplemented ones.

TABLE 4
Supplementation and stress treatment plan for
the in vitro skin equivalents.

| | Controls | | UV-Treatment | | Immune Cells | |
|---|---|---|---|---|---|---|
| | Non- | | Non- | | Non- | |
| | suppl. | Suppl. | suppl. | Suppl. | suppl. | Suppl. |
| Membrane | X | X | X | X | X | X |
| Equivalents | | | | | | |
| Collagen | X | X | X | X | | |
| Equivalents | | | | | | |

The supplemented and unsupplemented control equivalents were harvested after the end of the supplementation phase and prepared for cutting using a frozen section microtome. The irradiation of the skin equivalents was carried out after the supplementation phase in the “Dosimetersystem UV-AB-MAT” irradiation equipment made by Göbel UV Electronik GmbH. The irradiation dose was 20 J/cm² UVA. UVA (320-400 nm) generates reactive oxygen species via singlet oxygen, which can cause oxidative damage to the tissue and destroy it. The equivalents were cultivated for a further 24 hours after treatment and then prepared for the histological sections.

The generation of skin-infiltrating immunocomponents which in turn set off inflammatory processes is achieved by activation of the immune cells used. In order to be able to draw conclusions as to the effect of the substances on immune cells, in this test the immune cells were also treated with the active substances in addition to the equivalents. The immune cells were supplemented for 24 hours, activated and then “co-cultivated” for 3 days with the supplemented membrane equivalent. As in our experience the treatment of the collagen equivalents with the immune cells does not result in any visualisable effect because of the dermis component these models were excluded from this treatment.

3. Results

3.3 Histological Photographs

The results of the histological examinations are described below.

The membrane equivalents were stained with the haemalum/eosin staining which is suitable in this case for showing up the effects of the various treatments/stress induction on this model. Basically, regarding the membrane equivalents, it is found that the membrane detaches very easily during cutting with the frozen section microtome and cutting artefacts may occur, but they are easily identified. Moreover, the Tunel assay (TdT-mediated dUTP Nick End Labelling for detecting DNA strand breakages), which visualises apoptotic cells, cannot be carried out with the membrane equivalents as in this model nonspecific binding generates a high background which cannot be distinguished from the specific antigen-antibody binding.

On the one hand the collagen equivalents were stained with the haemalum/eosin stain and on the other hand apoptotic cells were visualised using the Tunel assay.

Membrane Equivalents

The membrane equivalents were subdivided into the following treatment groups:

TABLE 5
Supplementation and stress treatment plan of
the membrane equivalents.

| | Controls | | UV-Treatment | | Immune Cells | |
|---|---|---|---|---|---|---|
| | Non- | | Non- | | Non- | |
| | Suppl. | Suppl. | Suppl. | Suppl. | Suppl. | Suppl. |
| Membrane | X | X | X | X | X | X |
| Equivalents | | | | | | |

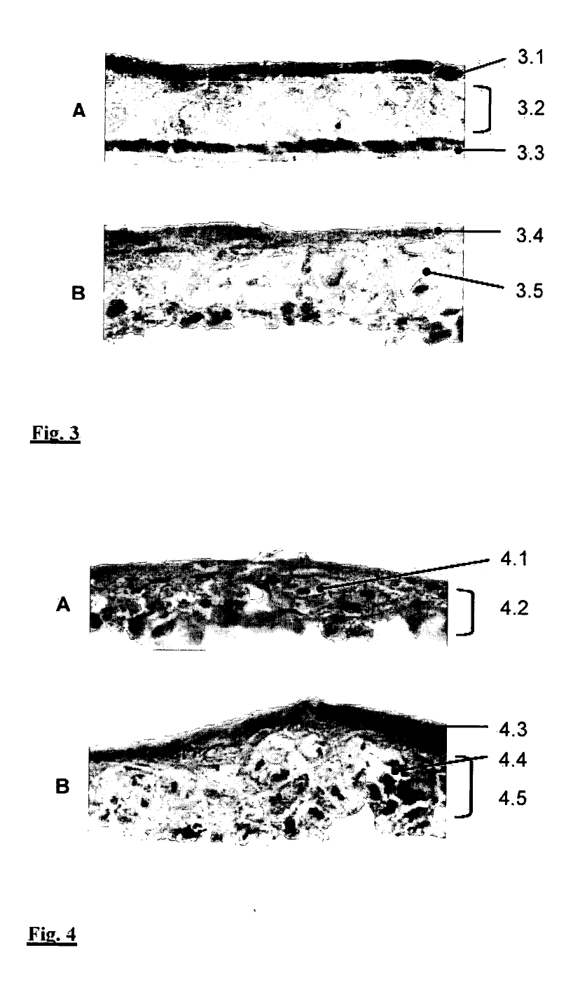

After the control equivalents have been cut and stained, first differences show up between the unsupplemented and supplemented model. Supplementation brings about sharper differentiation of the model, which can be seen from the thicker Stratum corneum (FIG. 3).

After UVA treatment, differences could be also be found between the unsupplemented and supplemented membrane equivalents. The keratinocytes in the unsupplemented UVA-irradiated model had an increased number of condensed cell nuclei in all cell layers, indicating increased apoptosis of the cells (FIG. 4a). This is also shown up by the colour change (orangey-red) and the poor discrimination of the individual cell layers.

Irradiation of the supplemented membrane equivalent, on the other hand, showed less drastic changes (FIG. 4b). The sharp differentiation of the keratinocytes (thickened Stratum corneum) can be seen here, and was also visible in the supplemented control equivalents. Moreover, a largely intact, proliferating keratinocyte layer can be seen with a few condensed cell nuclei.

In the “supplemented immune cells” treatment group the equivalents and in addition the immune cells were supplemented 24 hours before activation with the individual substances.

FIG. 5A very impressively shows increased “cluster” formation of the immune cells 24 hours after activation. A formation of the immune cells of this kind is an indication of successful stimulation of this type of cell, which can easily be checked under the microscope. The supplemented immune cells display very different characteristics in relation to the above mentioned cluster formation (FIG. 5B). There are a large number of individual cells and reduced aggregation of the cells, which strongly indicates an immunosuppressant and antiinflammatory potential of the bioactive ingredients used.

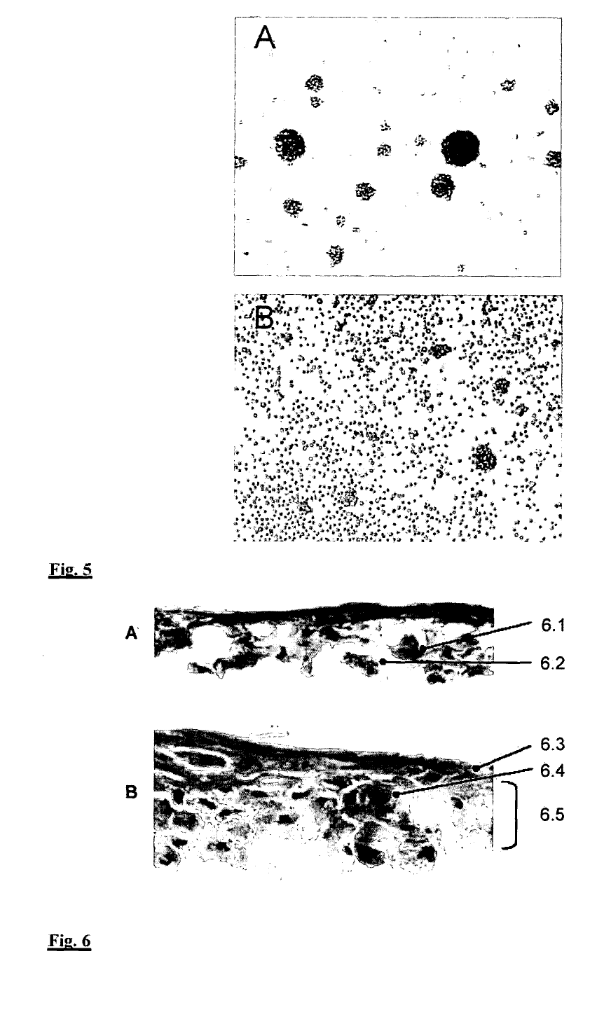

Comparable results can be observed after staining of the membrane equivalents. FIG. 6 shows the unsupplemented, immunostimulated (A) and supplemented, immunostimulated membrane equivalent (B). The sharply reduced integrity of the cells of the unsupplemented immunostimulated model should be noted. After supplementation and immunostimulation there is a substantially improved maintenance of the cell structure and hence a strengthening of the barrier intergity.

The collagen equivalents were subdivided into the following treatment groups (see Table 6) and after cutting investigated by histological (haemalum/eosin staining) and immunohistological methods (Tunel assay).

TABLE 6
Supplementation and stress treatment plan
of the collagen equivalents.

| | |
|---|---|
| Controls | UV-Treatment |

| | Unsuppl. | Suppl. | Unsuppl. | Suppl. |
|---|---|---|---|---|
| Collagen | X | X | X | X |
| Equivalents | | | | |

FIG. 7 shows, as a comparison, a cross section through normal skin. It is divided into the Stratum basale, Stratum spinosum, Stratum granulosum and Stratum corneum. In the Stratum basale the keratinocytes range from cubic to highly cylindrical in shape. The Stratum spinosum consists of 2-5 layers of polygonal, slightly flattened cells, whereas the keratinocytes of the Stratum granulosum have a flattened shape. In the Stratum corneum there are several layers of highly flattened, terminally differentiated keratinocytes, the corneocytes.

The structure of the collagen equivalent and the shape of the keratinocytes in the individual layers can readily be compared with the structure of the normal skin (FIG. 7B). It will immediately be realised that the cells of the Stratum basale to the Stratum corneum vary from a cylindrical shape to a flattened shape. By contrast, this model differs from normal skin in reduced differentiation of the keratinocytes to form the Stratum corneum and less creasing of the epidemis as a whole.

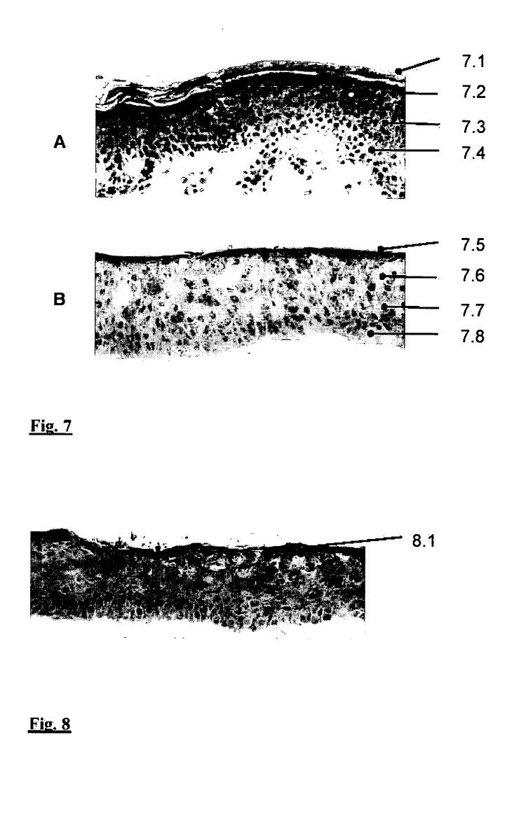

After supplementation, the overall staining, as in the membrane equivalents, shows sharper differentiation of the keratinocytes (FIG. 8).

The effects of UVA radiation on the apoptosis of the keratinocytes in the collagen equivalent was studied in more detail using the TUNEL assay. FIG. 9 shows the photographs of the control equivalents. In both cases (unsupplemented and supplemented) only individual apoptotic cells can be detected using this immuno-hystological method.

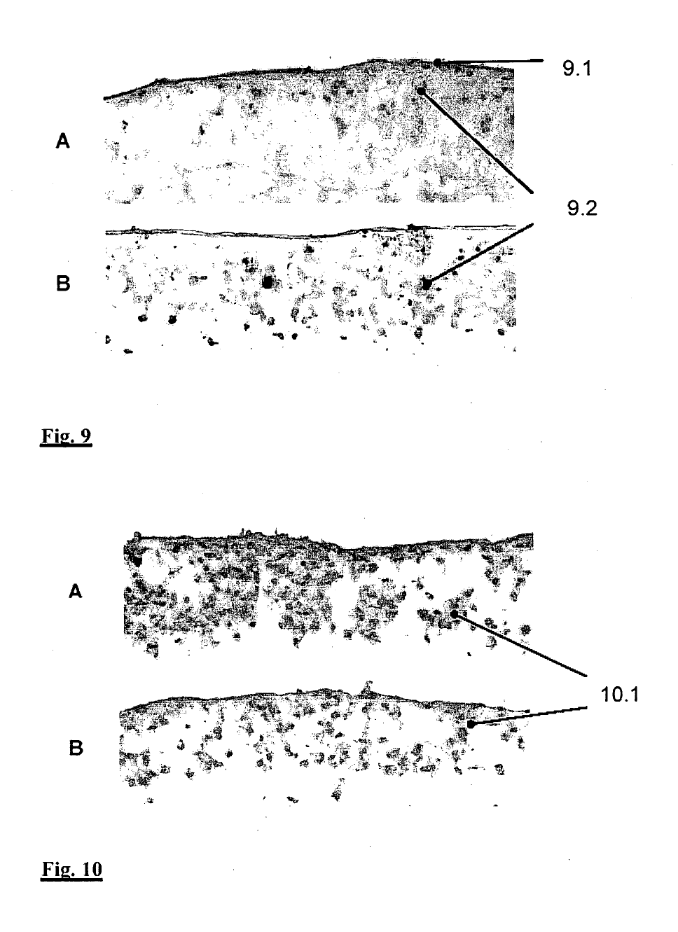

After UVA irradiation there is a sharp increase in the apoptotic cells, both in the unsupplemented and in the supplemented collagen equivalents, which is optically less noticeable in the supplemented model (FIG. 10).

The biologically active ingredients (standardised tomato extract, β-Carotene suspension, α-Tocopheryl acetate, standardized grape seed extract, ascorbic acid, selenium yeast) of Seresis® were first investigated in the above-mentioned amounts for possible cytotoxic effects and for their effect on the antioxidant capacity of human skin fibroblasts (NHDF). Apart from the pure mixture of active ingredients the entire contents of an active substance capsule were also used. In addition, a number of additives (oils, etc.) were also included in the test. Using two different in vitro skin models investigations were also carried out to see to what extent the protective effects of the active substances can be visualised. For this, the models were exposed to two different stressors (UV irradiation, activated immune cells).

The highest concentrations of the individual active substances in the tests carried out are above the average plasma values for the lipophilic substances (α-Tocopherol, lycopene, β-Carotene) but in a range which can be achieved by oral supplementation. In the case of ascorbic acid, on the other hand, the highest concentration is in a non-physiological range, and this is to be regarded as less critical in hydrophilic substances (Table 2).

After supplementation of the cells with the biologically active ingredients in the various dilutions, no or very few cytotoxic effects could be observed. The addition of the active substance mixture in all the dilutions used, on the contrary, even indicates that the human skin fibroblasts have an increased viability.

Using the capsule contents no cytotoxic effect was visible with regard to the mitochondrial activity but the lactate dehydrogenase in the cell culture supernatant was slightly raised. It is conceivable that the other ingredients of the capsule formulation, which are largely lipophilic by nature, interact with the cell membrane and thus affect the membrane fluidity. Generally, strongly lipophilic substances are always doubtful with regard to entry into aqueous cell culture media. For this reason only relatively low concentrations of the biologically active contents from the capsule could be introduced into the aqueous cell culture medium.

Measurement of the influence of the active substances on the antioxidant capacity of the cells produced very clear results. The biologically active ingredients exhibited a dosage-dependent increase in the antioxidant capacity. The antioxidant potential of all the dilutions tested was 145-160%, compared with the carrier control, significantly well above that of the reference substance α-Tocopherol (25 μM, 120%). Such an impressive increase in the antioxidant capacity of the cells was not achieved in this assay with isolated individual substances or even with combinations of substances (α-Tocopherol+ascorbic acid) to this degree (our own data). The results obtained here clearly illustrate the synergistic effect of the substances used in terms of their antioxidant effect.

Supplementation with the capsule contents also resulted in an increase in the antioxidant capacity of the cells which was comparable with that of α-Tocopherol. However, the comparatively large standard deviation of ±19% shows that the results with this formulation are not really reproducible, which is probably due to the above-mentioned problems with lipophilic substances in the cell culture.

The histological photographs of the in vitro skin models confirmed and added to the clear results obtained from the FluoroScan process.

Both in the membrane equivalents and in the collagen equivalents UVA irradiation was used as the stressor. UVA light (320-400 nm) acts primarily by generating reactive oxygen species. Reactive oxygen species are capable of oxidatively changing various substances found in the body. In addition to unsaturated fatty acids and cholesterol, nucleic acids, carbohydrates and proteins also present areas of attack for free radicals which may subsequently lead to cell function disorders and cell death. In the membrane equivalents, haemalum/eosin staining was used to show up the morphological changes in the cells. In addition, in the collagen equivalents, apoptotic cells were shown up using the TUNEL assay. In both models without supplementation UVA irradiation caused increased dying off of the keratinocytes, as expected. After supplementation, however, a largely intact and prolipherating keratinocyte layer could be detected in the membrane equivalent. In the case of the collagen equivalent a significant reduction in the number of apoptotic cells was observed. The thickening of the stratum corneum in both models after supplementation clearly indicates a strengthening of the barrier function, which may have a protective effect against the damaging influence of UVA irradiation, in addition to the antioxidant activity. It is conceivable that the addition of ascorbic acid (concentration for use: 0.68 mM) and the grapeseed extract in admixture is chiefly responsible for strengthening the epidermal barrier. In various studies both in vitro and in vivo using isolated individual substances similar observations were made. Moreover, tests on the effective different antioxidants such as α-Tocopherol, ascorbic acid, carotenoids, flavonoids, polyphenols, thiols and selenium exhibited partial protection against skin damage caused by exposure to UVA and/or UVB.

Activated immune cells, which as a consequence of activation release inflammation mediators (lymphokines) which in turn initiate inflammatory processes, were used as a further stressor for the membrane equivalents. In order to get as close as possible to an in vivo situation after oral supplementation, the immune cells were also treated with the active substances in the supplemented model. Surprisingly, after activation of the immune cells, an altered reaction was observed in the cells treated with the active substance mixture. The typical picture of “cluster” formation (an indication of successful activation) could only be observed fully in the untreated immune cells. In the supplemented cells there was a significantly reduced cluster formation and a large number of individual cells, strongly indicating an immuno-suppressant and anti-inflammatory potential. The extent to which this effect is subject to time limits and the precise mechanisms on which the effect is based (altered lymphokine pattern, increased cell growth, etc.) could not be explained at this stage.

The histological photographs of the membrane equivalents produced a comparable picture. Whereas after “co-culture” with the activated immune cells in the unsupplemented models there was a massive loss of cell integrity, a substantially improved maintenance of cell structure could be detected in the supplemented membrane equivalents. There are various possible mechanisms on which this observation might be based, such as for example the reduced activation of the immune cells or a direct anti-inflammatory protective effect of the active substances in the model.

The results obtained here on human skin fibroblasts and two different in vitro skin models make it possible to estimate the cytotoxicity and the antioxidant/anti-inflammatory effect of the active substance mixture under test in the living system. To summarise, it can be said that the concentrations of active substances used may be regarded as cytotoxically acceptable. A clear dosage-dependent increase in the antioxidant capacity of the cells was able to be achieved by supplementation with the bioactive ingredients of Seresis®. The histological photographs of the in vitro skin models show very clearly that the substance mix has not only an antioxidant potential (UVA irradiation) but also an anti-inflammatory potential (activated immune cells).

1. A method of protecting the skin of a person from UV rays, ageing under the effect of UV rays and/or inflammatory processes, which method comprises administering a composition containing synergistic amounts of antioxidants selected from the group comprising:
Vitamin E
Beta-carotene
Vitamin C
Selenium
an extract of Lycopersicum esculentum
an extract of Vitis vinifera, and
optionally a suitable carrier material to the said person in need thereof.

2. The method according to claim 1, wherein the composition consists of:
2 to 15 wt-% of vitamin E;
0.1 to 5 wt-% of beta-carotene;
20 to 60 wt-% of vitamin C;
5 to 20 wt-% of selenium;
5 to 20 wt-% of extract of Lycopersicum esculentum, and
5 to 20 wt-% of extract of Vitis vinifera;
based on the total amount of antioxidants.

3. The method according to claim 1, wherein vitamin E is used in the form of vitamin E acetate or vitamin E succinate.

4. The method according to one of claim 1, wherein vitamin C is used in the form of the sodium salt.

5. The method according to claim 1, wherein the carrier material is selected from the group consisting of natural vegetable oils, totally or partially hydrogenated vegetable oils, lecithins, plant phosphatides and natural waxes.

6. The method according to claim 5, wherein the carrier material is selected from the group consisting of soya oil, totally or partially hydrogenated soya oil, rapeseed oil, ground nut oil, soya lecithin, soya phosphatides, egg lecithin and beeswax.

7. The method according to claim 1, wherein the composition is used in the form of a soft or hard gelatine capsule, tablet, film-coated tablet, suppository or in a transdermal form.

8. The method according to claim 1, wherein the composition consists essentially of
15 to 45% by weight of antioxidants, and
55 to 85% by weight of carrier material.

9. The method according to claim 1, wherein the composition is administered orally or topically.

10. The method according to claim 1, wherein the composition is administered topically with at least one organic or inorganic UV filter.
